(19) United States
(12) Patent Application Publication
(10) Pub. No.: US 2003/0212388 A1
(43) Pub. Date: Nov. 13, 2003

(54) Treatment of localized pain with a flexible conformational array of light

(76) Inventor: Avigdor M. Ronn, Great Neck, NY (US)

Correspondence Address: Dr. Avigdor M. Ronn, 27A Bond Street, Great Neck, NY, 11021, US

(21) Appl. No.: 10/431,985

(22) Filed: May 8, 2003

Related U.S. Application Data
(60) Provisional application No. 60/379,982, filed on May 13, 2002.

(51) Int. Cl. A61B 18/18

(52) U.S. Cl. 606/9; 606/3

(57) ABSTRACT

Pain localized to a particular body area is treated by subjecting the affected body area to a flexible array of light emitting units that conforms to the contour of the affected body area, thereby delivering a therapeutic dose of light to that affected body area. A flexible array of light emitting units is conformingly placed onto the localized pain area. The light emitting units are energized to deliver a therapeutic dose of light over the localized pain area. Additionally, the same methodology is useful for treating facial wrinkles, remodeling facial and body collagen and providing holistic relaxation and stress relief.

This claims benefit of U.S. patent application Ser. No. 60/379,982, filed May 13, 2002.

### BACKGROUND OF THE INVENTION

The present invention relates to the treatment of localized pain by subjecting the pain affected area to low intensity light. In particular, the present invention is directed to the treatment of pain, localized to a body area, by subjecting the affected localized area to a flexible array of a plurality of light emitting units. The flexible array conforms to the contour of the affected body area, thereby delivering a therapeutic dose of light to that body area.

Laser biostimulation, also known as low level laser therapy (LILT) is a technique used to alleviate localized pain. Furthermore, the modality is equally effective to perform photorejuvenation, wrinkle removal, collagen tightening or remodeling, as well as general muscular and holistic relaxation. The therapy delivers 1-20 J/cm² of energy at low intensity (1-30 mW/cm²) to the area of the body requiring treatment. Presently, a low power laser is directed at an area for a predetermined time. Then, the laser is moved to an adjacent area and the laser is shone onto that area for the predetermined time. This process is repeated until the whole body area at which the pain is localized has been treated by the laser. Although the time that the laser is shone onto the patient for each spot may be only a few minutes, the total time can be uncomfortably long—particularly if the pain is localized over an extended area of the patient's body.

Thus, it would be, desirable to provide a method that can treat an extended area of localized pain without the need continually to move a single laser from spot to spot during treatment. This would alleviate the discomfort and inconvenience associated with the present dependence on the small spot size of conventional lasers.

Further, it would be desirable to provide an array of a plurality of light emitting units that can conform to an area of localized pain to treat conveniently that area without the need continually to move a single laser from spot to spot during treatment. The plurality of light emitting units can emit at any effective wavelength.

U.S. Pat. No. 6,096,066 describes a conformal patch for administering light therapy to subcutaneous tumors. U.S. Pat. No. 5,997,569 describes a flexible and adjustable grid for medical therapy.

### SUMMARY OF THE INVENTION

The present invention treats pain localized to a particular body area by subjecting the affected body area to a flexible array comprising a plurality of light emitting units. The flexible array can conveniently comprise a multitude of solid state lasers that substantially conform to the contour of the affected body area, thereby delivering a therapeutic dose of laser light to that affected body area. Thus, a flexible array of lasers is conformingly placed onto the localized pain area. The lasers are energized to deliver a therapeutic dose of laser light over the localized pain area. The lasers can emit at any convenient therapeutically effective wavelength.

The flexible array can comprise a plurality of light emitting diodes (“LED”) that substantially conforms to the contour of the affected body area, to deliver a therapeutic dose of electromagnetic energy in the form of light to that affected body area. The LEDs are energized to deliver a therapeutic dose of light over the localized area. The LEDs can emit at any convenient therapeutically effective wavelength.

### DETAILED DESCRIPTION OF THE INVENTION

The present invention treats pain that is localized over a particular body area by:

1) providing an array of light emitting diodes (“LED”) mounted in a supporting flexible medium such as, for example, an elastomeric polymer. In one embodiment, the flexible medium is a flexible circuit board material. The outputs of the LEDs are advantageously in the red to near infrared wavelengths to penetrate soft body tissue better. The output of the LEDs should overlap spatially to assure treatment of the entire pain affected body area, although it is not critical that the entire pain affected area be treated. As much as 40% of the localized pain affected area can be without light, although it is advantageous to cover at least 70% of the affected area. Even more advantageous, the LED light output should overlap each other to cover at least 85% of the affected area. The electrical requirements of the LEDs can be supplied by wire leads that connect to a power supply and a controller. The power supply can be battery or a transformer or a line voltage. The power output of the LED's should be controlled to yield about 1-30 mW/cm². Such LED units are commonly available. The localized pain area should be subjected to the LED output delivered until a predetermined energy total has been reached. For example, at an intensity of about 20 mW/cm² for about 8 minutes. The LED's can be incorporated into the devices described in U.S. Pat. No. 6,096,066 or U.S. Pat. No. 5,997,569.

2) Bringing the above array of LED's into conforming proximity to the pain affected body area;

3) Maintaining the array of LED's in a conforming proximity geometry to the pain affected body area; and

4) Subjecting the localized pain area to about 10 J/cm² of energy. As an example, the energy can be delivered at an intensity of about 20 mW/cm² for about 8 minutes.

The array of LED's can be maintained in a conforming proximity geometry to the pain affected body area by any convenient method such as, for example, holding the array, strapping the array, wrapping the array, clipping the array, zipping the array, or directing a force to maintain the LED array against the affected body area. Conveniently, the array can have a multiplicity of string used to tie the array against the affected body area. For the affected body being substantially in a horizontal relationship to the earth, the array can be laid onto the affected body area so that it is maintained in a proximate relationship to the affected body area by the force of gravity. Furthermore, the array can conveniently have a back surface that is tacky or “sticky” effective to maintain the array in a proximate relationship to the affected body area.

As described above, the localized pain area should be subjected to about 10 J/cm² of energy delivered, for example, at an intensity of about 20 mW/cm² for about 8 minutes. The energy can be in the range of about 7 J/cm² to about 12 J/cm². Advantageously, the energy should be between about 8.5 J/cm² to about 10 J/cm². Also, advantageously, the energy should be about 8 J/cm² to about 11 J/cm². Even more advantageously, the energy should be about 9.0 J/cm² to about 10 J/cm².

The intensity can be in the range of about 15 mW/cm² to about 25 mW/cm². The intensity should be below the intensity threshold that causes pain. For most people, that intensity threshold is about 150 mW/cm². Accordingly, the intensity should be below 150 mW/cm². The area should be subjected to the LED array's output for about 5 to about 10 minutes. For example, the area may be subjected to the light output for about 8 minutes.

The present invention also is directed to an array of LED's mounted in a supporting flexible medium. The supporting flexible medium can be, for example, an elastomeric polymer sheet, a network of flexible ribbons, a cloth quilting, a woven mat, or an interlocking network of solid sub-units similar to armor chain mail. The outputs of the LED's are advantageously in the red to near infrared wavelengths to penetrate soft body tissue better. The output of the LED's should overlap spatially to assure treatment of the entire pain affected body area, although it is not critical that the entire pain affected be treated. As much as 40% of the localized pain affected area can be without light, although it is advantageous to cover at least 70% of the affected area. Even more advantageous, the light output should overlap each other to cover at least 85% of the affected area. The electrical requirements of the LED's can be supplied by wire leads that connect to a power supply and a controller. The power supply can be battery, a transformer or a line voltage. The power output of the LED's should be controlled to yield about 1-30 mW/cm². It is advantageous that the LED's of the array be sized to deliver an intensity of about 20 mW/cm² for about 8 minutes.

The treatment method can be repeated as needed.

The present invention also treats pain localized over a particular body area by

1) providing an array of solid state lasers mounted in a supporting flexible medium such as, for example, an elastomeric polymer. The outputs of the lasers are advantageously in the red to near infrared wavelengths to penetrate soft body tissue better. It is convenient to use laser light at 630 nm, 635 nm, 652 nm, 665 nm, 670 nm, 690 nm, 740 nm, 830 nm, or 980 nm. It is advantageous to use the longer wavelengths of 690 nm, 740 nm, 830 nm, or 980 nm. The output of the lasers should overlap spatially to assure treatment of the entire pain affected body area, although it is not critical that the entire pain affected be treated. As much as 40% of the localized pain affected area can be without light, although it is advantageous to cover at least 70% of the affected area. Even more advantageous, the laser light output should overlap each other to cover at least 85% of the affected area. The electrical requirements of the lasers can be supplied by wire leads that connect to a power supply and a controller. The power supply can be battery, a transformer or a line voltage. The power output of the lasers should be controlled to yield about 1-150 mW/cm². It is advantageous that the solid-state lasers of the array are sized to subject the localized pain area to about 9.6 J/cm² of energy delivered at an intensity of about 20 mW/cm² for about 8 minutes. Such lasers can be obtained from Boston Laser, Hitachi, Lumenis, or Panasonic Corp. The lasers can be incorporated into the devices described in U.S. Pat. No. 6,096,066 or U.S. Pat. No. 5,997,569.

2) Bringing the above array of solid-state lasers into conforming proximity to the pain affected body area;

3) Maintaining the array of solid-state lasers in a conforming proximity geometry to the pain affected body area; and

4) Subjecting the localized pain area to about 9.6 J/cm² of laser energy. As an example, the energy can be delivered at an intensity of about 20 mW/cm² for about 8 minutes.

The array of solid-state lasers can be maintained in a conforming proximity geometry to the pain affected body area by any convenient method such as, for example, holding the array, strapping the array, wrapping the array, clipping the array, zipping the array, or directing a force to maintain the solid-state laser array against the affected body area. Conveniently, the array can have a multiplicity of string used to tie the array against the affected body area. In the case of the affected body being substantially in a horizontal relationship to the earth, the array can be laid onto the affected body area so that it is maintained in a proximate relationship to the affected body area by the force of gravity.

As described above, the localized pain area may be subjected to about 9.6 J/cm² of energy delivered at, for example, an intensity of about 20 mW/cm² for about 8 minutes. The energy can be in the range of about 7 J/cm² to about 12 J/cm². Advantageously, the energy should be between about 8.5 J/cm² to about 10 J/cm². Also, advantageously, the energy should be about 8 J/cm² to about 11 J/cm². Even more advantageously, the energy should be about 9.0 J/cm² to about 10 J/cm².

Conveniently, the intensity should be from about 1 mW/cm² to about 150 mW/cm². Advantageously, the intensity can be about 15 mW/cm² to about 25 mW/cm². The area should be subjected to the laser array's laser output for about 8 minutes. Advantageously, the area should be subjected to the light output for about 5 to about 10 minutes.

Further, it is convenient to use laser light at the wavelength between about 600 nm and about 1000 nm. It is particularly convenient to use laser light at the wavelength of 630 nm, 635 nm, 652 nm, 665 nm, 670 nm, 690 nm, 740 nm, 830 nm, or 980 nm. It is advantageous to use laser light at the wavelength of 690 nm, 740 nm, 830 nm, or 980 nm.

The present invention also is directed to an array of solid state lasers mounted in a supporting flexible medium. The supporting flexible medium can be, for example, an elastomeric polymer sheet, a network of flexible ribbons, a cloth quilting, a woven mat, or an interlocking network of solid sub-units similar to armor chain mail. The output of the lasers is advantageously in the red to near infrared wavelengths to penetrate soft body tissue better. It is advantageous to use laser light at 630 nm, 635 nm, 652 nm, 665 nm, 670 nm, 690 nm, 740 nm, 830 nm, or 980 nm. It is especially advantageous to use the long wavelength of 690 nm, 740 nm, 830 nm, 980 nm. The output of the lasers should overlap spatially to assure treatment of the entire pain affected body area, although it is not critical that the entire pain affected be treated. As much as 40% of the localized pain affected area can be without light, although it is advantageous to cover at least 70% of the affected area. Even more advantageous, the laser light output should overlap each other to cover at least 85% of the affected area. The electrical requirements of the lasers can be supplied by wire leads that connect to a power supply and a controller. The power supply can be battery or line. The power output of the lasers should be controlled to yield about 1-30 mW/cm². It is advantageous that the solid-state lasers of the array are sized to subject the localized pain area to about 9.6 J/cm² of energy delivered at an intensity of about 20 mW/cm² for about 8 minutes.

The treatment method can be repeated as needed.

The LILT method of the present invention has been tested in over one hundred persons with a variety of musculoskeletal pains. Pain alleviation was found on an average to drop approximately 2 units on a standard pain 10 scale in which 10 is the worst pain per single treatment. These treatments included but were not limited to patients suffering from sore lower backs, tennis elbow, golf elbow, tendonitis, bursitis, sciatica, sore upper back and facial pain.

Some specific examples of non-standard pain and a discussion of specific examples of photorejuvenation (wrinkle reduction and general toning of the underlying structural collagen), at for example the face, by lasers and LED sources are described below.

Eight patients were treated for stress induced Temporal Mandibular Joint (TMJ) syndrome with very good results, reporting on average a relief of approximately 2 units on the standard pain 10 scale for each treatment. These patients have been previously treated by dentists via nerve blocking injections or had been fitted with mechanical devices altering their bite profile with limited success.

The LILT method of the present invention was tested for effectiveness in skin rejuvenation and wrinkle reduction in 12 patients, ranging in age from 30 to 65. 90% of the treated patients manifested a significant reduction in deep wrinkle depth profile. Photography, performed before and after each treatment, revealed that fine wrinkles smoothing was evident within one hour of treatment. Smoothing means a reduction of about 10% or more in the number of wrinkles or the shortening of wrinkle lines of about 10% or more. Also, the nasolabial areas showed significant depth reduction.

Younger patients reported a tightening and a visually fuller lips profile. Photography confirmed that latter finding in three cases. Furthermore, the overall facial treated skin texture was subjectively defined by all 12 as being “silkier” and “smoother.” Magnified views of the treated areas confirmed these textural changes.

The LILT method of the present invention was tested for collagen remodeling in 7 patients. Fine line erasure was evident in 70% of the treatment cohort in the perioral and upper lip areas. Additionally, 30% of the cohort reported their lips feeling and looking “plumper’ and more robust.

Collagen remodeling is a light based technology, which is rapidly gaining acceptance with men and women who are not willing to undergo invasive surgical procedures. A simple and non- invasive procedure delivered in a relatively short time by a variety of laser and LED sources, the technique is finding a wide audience in both facial plastics and dermatological offices. The flexible conformable LED array was found to be as effective as the laser procedure in the 7 patients mentioned above. Results were followed via both photography and by subjective patient self evaluation reports.

The LILT method of the present invention was tested on three patients for pain associated with wound healing. One had a broken arm and a broken hand. Treatment of five sessions over two weeks significantly reduced the pain prior to treatment. The second was treated after a double extraction dental procedure. Two treatments in three days alleviated the pain and significantly reduced facial edema and puffiness. The pain was reported as 9 on the pain 10 scale pretreatment and as a 3 after treatment.

The third was treated for pain following a left rotator cuff surgery. Pretreatment pain was reported as a 9. After six treatment sessions delivered over a two week period, the patient reported significant mobility in a nearly immobile shoulder and a pain scale self evaluation of 3.

Acne can also be treated by the method of the present invention. The medical community is moving away from the pharmaceutical medications to light based therapy for the treatment of acne mainly because of the significant morbidity of the side effects associated with the acne medications.

There are two purely light based approaches to the treatment that are finding wide acceptance. One relies on a thermal alteration of the sebaceous gland by a deeply penetrating laser (1450 nm) providing heat to the lesion. The other relies on blue or near UV light destruction of single or tightly clustered facial lesions (400-420 nm). The LILT technique described here is able to deliver both modalities. The flexible LED array is perceived to be particularly effective as is can be adapted to any wavelength of choice.

It is apparent that certain wavelengths may be more efficacious than other wavelengths. The present invention includes the use of any efficacious wavelength as would be known or revealed by experiments commonly performed in the art.

The present invention was also tested as a method for providing muscular and holistic relaxation resulting from stress. The LILT and LED based LILT both provide a safe non-invasive and gentle relief in all five patients treated. Upon the completion of each treatment, a follow-up questionnaire was filled by the patients to evaluate progress. All patients polled reported feeling relaxed and even sleepy after a single 30 minute session. They reported overall relaxation (holistic relaxation) as well as specific relaxation of specifically tense muscles (muscular relaxation).

Thus, the present invention provides a method of photorejuvenation localized over a particular body area comprising the steps of: a. providing an array of light emitting diodes (LEDs) mounted in a supporting flexible medium; b. bringing said array of LEDs into conforming proximity to the body area; c. maintaining said array of LEDs in a conforming proximity geometry to the body area; and d. subjecting the body area to about 7-12 J/cm² of energy by energizing the LEDs.

The present invention also provides a method of photorejuvenation over a particular body area comprising the steps of: a. providing an array of solid state lasers mounted in a supporting flexible medium; b. bringing said array of solid-state lasers into conforming proximity to the body area; c. maintaining said array of solid-state lasers in a conforming proximity geometry to the body area; and d. subjecting the localized pain area to about 7-12 J/cm² of energy from the lasers.

The present invention also provides a method of wrinkle removal localized over a particular body area comprising the steps of: a. providing an array of light emitting diodes (LEDs) mounted in a supporting flexible medium; b. bringing said array of LEDs into conforming proximity to the body area; c. maintaining said array of LEDs in a conforming proximity geometry to the body area; and d. subjecting the body area to about 7-12 J/cm² of energy by energizing the LEDs.

The present invention also provides a method of wrinkle removal over a particular body area comprising the steps of: a. providing an array of solid state lasers mounted in a supporting flexible medium; bringing said array of solid-state lasers into conforming proximity to the body area; c. maintaining said array of solid-state lasers in a conforming proximity geometry to the body area; and d. subjecting the localized pain area to about 7-12 J/cm² of energy from the lasers.

The present invention also provides a method of collagen remodeling localized over a particular body area comprising the steps of: a. providing an array of light emitting diodes (LEDs) mounted in a supporting flexible medium; b. bringing said array of LEDs into conforming proximity to the body area; c. maintaining said array of LEDs in a conforming proximity geometry to the body area; and d. subjecting the body area to about 7-12 J/cm² of energy by energizing the LEDs.

The present invention also provides a method of collagen remodeling over a particular body area comprising the steps of: a. providing an array of solid state lasers mounted in a supporting flexible medium; b. bringing said array of solid-state lasers into conforming proximity to the body area; c. maintaining said array of solid-state lasers in a conforming proximity geometry to the body area; and d. subjecting the localized pain area to about 7-12 J/cm² of energy from the lasers.

The present invention also provides a method of muscular or holistic relaxation localized over a particular body area comprising the steps of: a. providing an array of light emitting diodes (LEDs) mounted in a supporting flexible medium; b. bringing said array of LEDs into conforming proximity to the body area; c. maintaining said array of LEDs in a conforming proximity geometry to the body area; and d. subjecting the body area to about 7-12 J/cm² of energy by energizing the LEDs.

The present invention also provides a method of muscular relaxation or holistic relaxation over a particular body area comprising the steps of: a. providing an array of solid state lasers mounted in a supporting flexible medium; b. bringing said array of solid-state lasers into conforming proximity to the body area; c. maintaining said array of solid-state lasers in a conforming proximity geometry to the body area; and d. subjecting the localized pain area to about 7-12 J/cm² of energy from the lasers.

What is claimed is:

1. A method of treating pain localized over a particular body area comprising the steps of:
providing an array of light emitting diodes (LEDs) mounted in a supporting flexible medium;
bringing said array of LEDs into conforming proximity to the pain affected body area;
maintaining said array of LEDs in a conforming proximity geometry to the pain affected body area; and
subjecting the localized pain area to about 7-12 J/cm² of energy by energizing the LEDs.

2. A method of treating pain localized over a particular body area comprising the steps of:
providing an array of solid state lasers mounted in a supporting flexible medium;
bringing said array of solid-state lasers into conforming proximity to the pain affected body area;
maintaining said array of solid-state lasers in a conforming proximity geometry to the pain affected body area; and
subjecting the localized pain area to about 7-12 J/cm² of energy from the lasers.

3. The method of claim 1, wherein the localized pain area is subjected to about 8.5 to about 10 J/cm² of energy.

4. The method of claim 1, wherein the localized pain area is subjected to about 9 to about 10 J/cm² of energy.

5. The method of claim 1, wherein the localized pain area is subjected to energy intensity of about 15 to about 25 mW/cm² for a duration of about 5 to about 10 minutes.

6. The method of claim 1, wherein the output of the LEDs is in the wavelength region from about 500 nm to about 1000 nm.

7. The method of claim 2, wherein the localized pain area is subjected to about 8.5 to about 10 J/cm² of energy.

8. The method of claim 2, wherein the localized pain area is subjected to about 9 to about 10 J/cm² of energy.

9. The method of claim 2, wherein the localized pain area is subjected to energy intensity of about 15 to about 25 mW/cm² for a duration of about 5 to about 10 minutes.

10. The method of claim 2, wherein the laser output wavelength is from about 600 nm to about 1000 nm.

11. The method of claim 2, wherein the laser output wavelength is 630 nm, 635 nm, 652 nm, 665 nm, 670 nm, 740 nm, 830 nm, or 980 nm.

12. A method of photorejuvenation localized over a particular body area comprising the steps of:
providing an array of light emitting diodes (LEDs) mounted in a supporting flexible medium;
bringing said array of LEDs into conforming proximity to the body area;
maintaining said array of LEDs in a conforming proximity geometry to the body area; and
subjecting the body area to about 7-12 J/cm² of energy by energizing the LEDs.

13. A method of photorejuvenation over a particular body area comprising the steps of:
providing an array of solid state lasers mounted in a supporting flexible medium;
bringing said array of solid-state lasers into conforming proximity to the body area;
maintaining said array of solid-state lasers in a conforming proximity geometry to the body area; and
subjecting the localized pain area to about 7-12 J/cm² of energy from the lasers.

14 A method of wrinkle removal localized over a particular body area comprising the steps of:
providing an array of light emitting diodes (LEDs) mounted in a supporting flexible medium;
bringing said array of LEDs into conforming proximity to the body area;
maintaining said array of LEDs in a conforming proximity geometry to the body area; and
subjecting the body area to about 7-12 J/cm² of energy by energizing the LEDs.

15. A method of wrinkle removal over a particular body area comprising the steps of:
providing an array of solid state lasers mounted in a supporting flexible medium;
bringing said array of solid-state lasers into conforming proximity to the body area;
maintaining said array of solid-state lasers in a conforming proximity geometry to the body area; and
subjecting the localized pain area to about 7-12 J/cm² of energy from the lasers.

16 A method of collagen remodeling localized over a particular body area comprising the steps of:
providing an array of light emitting diodes (LEDs) mounted in a supporting flexible medium;
bringing said array of LEDs into conforming proximity to the body area;
maintaining said array of LEDs in a conforming proximity geometry to the body area; and
subjecting the body area to about 7-12 J/cm² of energy by energizing the LEDs.

17. A method of collagen remodeling over a particular body area comprising the steps of:
providing an array of solid state lasers mounted in a supporting flexible medium;
bringing said array of solid-state lasers into conforming proximity to the body area;
maintaining said array of solid-state lasers in a conforming proximity geometry to the body area; and
subjecting the localized pain area to about 7-12 J/cm² of energy from the lasers.

18 A method of muscular or holistic relaxation localized over a particular body area comprising the steps of:
providing an array of light emitting diodes (LEDs) mounted in a supporting flexible medium;
bringing said array of LEDs into conforming proximity to the body area;
maintaining said array of LEDs in a conforming proximity geometry to the body area; and
subjecting the body area to about 7-12 J/cm² of energy by energizing the LEDs.

19. A method of muscular relaxation or holistic relaxation over a particular body area comprising the steps of:
providing an array of solid state lasers mounted in a supporting flexible medium;
bringing said array of solid-state lasers into conforming proximity to the body area;
maintaining said array of solid-state lasers in a conforming proximity geometry to the body area; and
subjecting the localized pain area to about 7-12 J/cm² of energy from the lasers.
